(19) United States
(12) Patent Application Publication
(10) Pub. No.: US 2025/0218560 A1
(43) Pub. Date: Jul. 3, 2025

(54) SYSTEM FOR GENERATING PERSONALIZED ORAL CARE PLANS FOR ENHANCED ORAL HEALTH AND DISEASE PREVENTION

(71) Applicants: Paul Andrew Dominique, Atlanta, GA (US); Mina Amir Youssef, Mableton, GA (US)

(72) Inventor: Paul Andrew Dominique, Atlanta, GA (US)

(21) Appl. No.: 19/063,387

(22) Filed: Feb. 26, 2025

(51) Int. Cl. G16H 20/00; A46B 15/00; A61B 5/00; A61B 10/00

(52) U.S. Cl. CPC G16H 20/00; A46B 15/0008; A46B 2200/1066; A61B 5/0088; A61B 10/0051

(57) ABSTRACT

The present invention provides a system for enhanced oral health and disease prevention. The system comprises a computing device configured to execute modules, such as a tooth-brushing data module to assess brushing frequency and duration, an oral microbiome analysis module for analyzing saliva samples, a plaque assessment module to evaluate plaque levels through images or videos, a breath analysis module for assessing oral odor of the user, a data triangulation module to generate an oral health scorecard, and a preventive care recommendation module to create personalized oral care plans that include tailored schedules for professional cleaning appointments. The system includes a smart contract module to enforce compliance with the personalized oral care plans, thereby offering incentives or cost adjustments. The system promotes improved oral hygiene practices, early detection of oral diseases, and proactive dental care management.

### FIELD OF THE INVENTION

The present disclosure relates generally to oral health management systems and, more particularly, to a system for generating personalized oral care plans using data collected from smart toothbrushes, saliva testing devices, digital applications and breath analyzers, thereby enhancing oral health and preventing dental diseases through compliance-based preventive care.

### BACKGROUND

Oral health is an integral component of overall health, yet it remains one of the most neglected aspects of preventive healthcare. Conventional approaches to dental care predominantly focus on treatment rather than prevention, which requires individuals to visit dental clinics only after developing symptoms such as cavities, gum diseases, or other oral health problems. These methods largely depend on the patient's compliance with recommended oral hygiene practices, such as regular tooth-brushing and flossing. However, studies and clinical experience suggest that adherence to these practices is inconsistent among patients, thereby leading to the development of biofilm and plaque, which are primary contributors to dental diseases.

In recent years, various inventions have emerged to encourage better oral hygiene practices. For instance, electric toothbrushes with built-in timers and pressure sensors have aimed to improve brushing habits by ensuring users brush for the recommended duration and avoid excessive force. Similarly, smartphone-connected toothbrushes provide real-time feedback on brushing techniques. While these advancements have made some strides in promoting awareness, they primarily address the mechanics of tooth-brushing and fail to provide a comprehensive view of the user's oral health or account for factors such as the microbiological profile of the mouth.

Another category of prior art focuses on plaque detection using imaging technologies. For example, some smartphone apps and devices utilize specialized cameras to detect plaque on teeth, which provides the users with visual feedback on areas requiring attention. However, these solutions are limited to anterior teeth visible within the imaging field and cannot assess plaque in hard-to-reach areas. Moreover, these systems depend heavily on the user's willingness and ability to act on the feedback, which often falls short in achieving significant behavioral change.

Saliva testing kits have also been introduced as a diagnostic tool to assess oral microbiology. These kits analyze saliva samples to detect the presence of pathogenic bacteria linked to dental diseases. While effective in identifying potential risks, these systems often operate in isolation and do not integrate data from other sources, such as tooth-brushing patterns or plaque imaging, to create a holistic understanding of oral health. Furthermore, the high cost and limited accessibility of such kits can deter widespread adoption.

Despite these technological advancements, the limitations of prior art remain evident. None of the existing systems effectively address the root cause of oral health issues, which is the inability of most individuals to fully adhere to optimal oral care practices. Moreover, these systems fail to provide a tailored preventive care plan or incentivize compliance with regular dental hygiene appointments. As a result, patients continue to face costly therapeutic treatments for preventable dental diseases.

To address these limitations, there is a need for a system for generating personalized oral care plans using data collected from smart toothbrushes, saliva testing devices, digital applications and breath analyzers, thereby enhancing oral health and preventing dental diseases through compliance-based preventive care. There is also a need for a system that integrates oral microbiological data, brushing patterns, plaque scoring and oral odor levels to provide a comprehensive understanding of a user's oral health and create tailored preventive strategies. Further, there is also a need for a system that incentivizes user compliance with professional hygiene appointments while dynamically adjusting care plans based on real-time user data, thereby ensuring affordability and accessibility without requiring expensive specialized equipment.

### SUMMARY OF THE INVENTION

The following presents a simplified summary of one or more embodiments of the present disclosure in order to provide a basic understanding of such embodiments. This summary is not an extensive overview of all contemplated embodiments, and is intended to neither identify key nor critical elements of all embodiments, nor delineate the scope of any or all embodiments.

The present disclosure, in one or more embodiments, relates to a system for generating personalized oral care plans using data collected from smart toothbrushes, saliva testing devices, digital applications and breath analyzers, thereby enhancing oral health and preventing dental diseases through compliance-based preventive care. The system integrates oral microbiological data, brushing patterns, plaque scoring and oral odor levels to provide a comprehensive understanding of a user's oral health and create tailored preventive strategies. The system incentivizes user compliance with professional hygiene appointments while dynamically adjusting care plans based on real-time user data, thereby ensuring affordability and accessibility without requiring expensive specialized equipment.

In one embodiment herein, the system comprises a computing device having a processor and a memory to store one or more instructions executable by the processor. The computing device is configured to execute a plurality of modules, which includes a tooth-brushing data module, an oral microbiome analysis module, a plaque assessment module, a breath analysis module, a data triangulation module, and a preventive care recommendation module.

In one embodiment herein, the tooth-brushing data module is configured to collect data related to brushing frequency and duration from at least one smart toothbrush used by the user. In one embodiment herein, the at least one smart toothbrush is a manually operated toothbrush equipped with a clip-on attachment that monitors and records the brushing frequency and duration.

In one embodiment herein, the oral microbiome analysis module is configured to collect data related to microbiological characteristics and pathogenic bacteria analyzed from a saliva sample of the user. In one embodiment herein, the saliva sample is collected by the user in a vial provided by a saliva testing device and subsequently analyzed in a laboratory to identify microbiological characteristics and detect the presence of pathogenic bacteria.

In one embodiment herein, the plaque assessment module is configured to collect data related to plaque scoring generated through a digital application. In one embodiment herein, the digital application uses one or more advanced artificial intelligence (AI)-based image processing models that are configured to analyze one or more images or videos of the user's teeth captured by at least one capturing unit to generate the plaque scoring, which evaluates effectiveness of the user's tooth-brushing technique. The at least one capturing unit includes a camera, a smartphone, a webcam, an intraoral camera, an intraoral scanner, a handheld imaging device and thereof.

In one embodiment herein, the breath analysis module is configured to collect data related to oral odor of the user by detecting volatile sulfur compounds (VSCs) and other biomarkers using a breath analyzer. In one embodiment herein, the data triangulation module is configured to gather and process the data from the tooth-brushing data module, oral microbiome analysis module, plaque assessment module and breath analysis module to generate an oral health scorecard for the user that provides insights into the user's oral health status.

In one embodiment herein, the preventive care recommendation module is configured to generate a personalized oral care plan based on the oral health scorecard of the user. The personalized oral care plan includes a tailored schedule of professional cleaning appointments with dental hygienists, thereby enhancing oral health and disease prevention.

In one embodiment herein, the plurality of modules further comprises a smart contract module that is configured to enforce user's compliance with the personalized oral care plan by automatically monitoring and verifying the user's submission of data from the at least one smart toothbrush, at least one saliva testing device, a digital application, breath analyzer and user's attendance percentage at scheduled professional cleaning appointments. The smart contract module is further configured to adjust cost of the personalized oral care plan based on the user's compliance.

In one embodiment herein, the computing device is configured to communicate with a server and a user device via a network. The computing device and user device include at least one of a computer, a smartphone, a laptop, a tablet, and thereof.

According to an aspect, a method is disclosed for operating the system for enhanced oral health and disease prevention using the computing device. First, at one step, the tooth-brushing data module collects the data related to the brushing frequency and duration from the at least one smart toothbrush used by the user. At another step, the oral microbiome analysis module collects the data related to microbiological characteristics and pathogenic bacteria from the at least one saliva testing device. At another step, the plaque assessment module collects the data related to the plaque scoring generated through the digital application. At another step, the breath analysis module collects the data related to oral odor of the user by detecting volatile sulfur compounds (VSCs) and other biomarkers using the breath analyzer.

At another step, the data triangulation module gathers and processes the data from the tooth-brushing data module, oral microbiome analysis module, plaque assessment module and breath analysis module to generate an oral health scorecard for the user that provides insights into the user's oral health status. Further, at another step, the preventive care recommendation module generates the personalized oral care plan based on the oral health scorecard of the user.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

The accompanying drawings, which are incorporated in and constitute a part of the specification, illustrate an embodiment of the invention, and, together with the description, explain the principles of the invention.

FIG. 1 illustrates a block diagram of a system for enhanced oral health and disease prevention, in accordance with embodiments of the invention.

FIG. 2 illustrates a flowchart of a method for operating the system for enhanced oral health and disease prevention, in accordance with embodiments of the invention.

### DETAILED DESCRIPTION

Reference will now be made in detail to the present preferred embodiments of the invention, examples of which are illustrated in the accompanying drawings. Wherever possible, the same reference numerals are used in the drawings and the description to refer to the same or like parts.

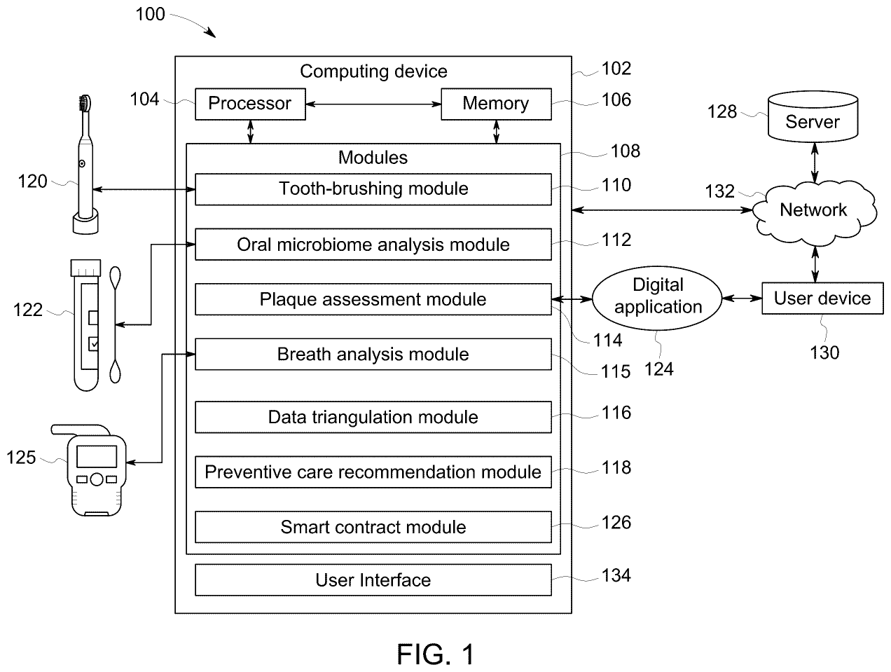

FIG. 1 refers to a block diagram of a system 100 for enhanced oral health and disease prevention. In one embodiment herein, the system 100 generates personalized oral care plans using data collected from one or more external devices, thereby enhancing oral health and preventing dental diseases through compliance-based preventive care. The system 100 integrates oral microbiological data, brushing patterns, plaque scoring and oral odor levels to provide a comprehensive understanding of a user's oral health and create tailored preventive strategies. The system 100 incentivizes user compliance with professional hygiene appointments while dynamically adjusting care plans based on real-time user data, thereby ensuring affordability and accessibility without requiring expensive specialized equipment.

In one embodiment herein, the system 100 comprises a computing device 102 equipped with a processor 104 and a memory 106, where the memory 106 is configured to store one or more instructions executable by the processor 104. The computing device 102 is configured to operate a plurality of modules 108, each performing specific functions to facilitate oral health monitoring and disease prevention. These modules 108 include a tooth-brushing data module 110, an oral microbiome analysis module 112, a plaque assessment module 114, a breath analysis module 115, a data triangulation module 116, and a preventive care recommendation module 118.

In one embodiment herein, the tooth-brushing data module 110 is configured to monitor and record data related to frequency and duration of the user's brushing activity. This data is collected using at least one smart toothbrush 120. The smart toothbrush 120 may include, but not limited to, an electric or manually operated toothbrush. In one specific implementation, the smart toothbrush 120 is a manually operated device equipped with a detachable clip-on attachment that incorporates one or more sensors to track brushing movements, monitor time spent on brushing, and store or transmit this data for further analysis. The collected data assists in identifying the user's brushing habits, thereby enabling targeted recommendations for improving oral hygiene.

In one embodiment herein, the oral microbiome analysis module 112 is configured to collect and analyze data related to microbiological characteristics and the presence of pathogenic bacteria in the user's oral cavity. This data is obtained from at least one saliva testing device 122, which includes a vial for collecting a saliva sample from the user. The vial may incorporate scaling mechanisms to ensure sample integrity during handling and transportation. The saliva testing device 122 further facilitates the microbiological analysis of the collected sample, thereby enabling the identification of microbial diversity, harmful pathogens, and any imbalances in the oral microbiome. This analysis detects early signs of dental diseases or infections, thereby aiding in timely interventions.

In one embodiment herein, the plaque assessment module 114 is configured to assess the presence and extent of plaque on the user's teeth based on the plaque scoring generated by a digital application 124. The digital application 124 could use advanced artificial intelligence (AI)-based image processing models that analyze one or more images or videos of the user's teeth. These images or videos are captured using at least one capturing unit, which may include, but not limited to, a camera, a smartphone, a webcam, an intraoral camera, an intraoral scanner, or a handheld imaging device. The plaque assessment module 114 evaluates the user's tooth-brushing technique based on the plaque scoring, which measures the effectiveness of plaque removal during daily brushing. This real-time feedback enables the user to identify areas requiring additional attention, thus promoting better oral hygiene practices. In one embodiment herein, the digital application 124 could be, but not limited to, a software application, a web application, a smartphone application, a web extension, a user interface application, and thereof.

In one embodiment herein, the breath analysis module 115 is configured to collect data related to oral odor of the user by detecting volatile sulfur compounds (VSCs) and other biomarkers using a breath analyzer 125. The breath analysis module 115 integrates artificial intelligence (AI)-powered pattern recognition models to correlate breath odor data with potential oral health conditions, such as periodontal disease, dry mouth (xerostomia), bacterial overgrowth, or systemic health issues such as diabetes and gastrointestinal disorders. The system 100 applies machine learning models that compare the detected VSC levels against a baseline odor profile, thereby enabling early identification of anomalies that may require intervention.

In one embodiment herein, the data triangulation module 116 is configured to integrate and process the data collected by the tooth-brushing data module 110, the oral microbiome analysis module 112, the plaque assessment module 114 and the breath analysis module 115. By consolidating these data, the data triangulation module 116 generates an oral health scorecard for the user. This oral health scorecard provides a comprehensive overview of the user's oral health status, which highlights key metrics such as brushing efficiency, microbiome balance, plaque and oral odor levels. The triangulation of diverse datasets ensures a holistic evaluation of oral health, thereby enabling accurate and actionable insights for the user.

In one embodiment herein, the preventive care recommendation module 118 is configured to generate a personalized oral care plan for the user based on the oral health scorecard. This personalized oral care plan includes specific recommendations tailored to the user's unique oral health needs. For example, the personalized oral care plan may outline a tailored schedule of professional cleaning appointments with dental hygienists, thereby providing detailed timelines and reminders. By addressing the user's specific oral health concerns, the personalized oral care plan aids in preventing dental diseases, thereby maintaining optimal oral hygiene, and improving overall oral health outcomes.

In one embodiment herein, the computing device 102 further comprises a user interface 134 that is configured to enhance user interaction and facilitate seamless operation of the system 100. The user interface 134 allows users to access, monitor, and manage various features of the system 100, such as viewing their oral health scorecard, brushing data, microbiome analysis results, plaque assessment outcomes and oral odor data. The user interface 134 is intuitive and user-friendly, thereby offering functionalities such as real-time notifications, reminders for brushing or scheduled professional cleaning appointments, and visual feedback on brushing techniques. Additionally, the user interface 134 enables the user to customize their personalized oral care plan based on the insights generated by the system 100. In one embodiment herein, the user interface 134 can be a touch screen.

In one embodiment herein, the modules 108 further comprise a smart contract module 126 that is configured to promote user compliance with the personalized oral care plan. This smart contract module 126 automatically monitors and verifies the user's adherence by tracking data submissions from the smart toothbrush 120, the saliva testing device 122, the digital application 124, the breath analyzer 125 and user's attendance percentage at scheduled professional cleaning appointments. Using predefined conditions, the smart contract module 126 can dynamically adjust the cost of the personalized oral care plan, thereby offering incentives or imposing penalties based on user's compliance. For instance, the users demonstrating consistent adherence may receive reduced costs or rewards, thereby encouraging sustained participation in the oral care regimen.

In one embodiment herein, the computing device 102 is configured to communicate seamlessly with a server 128 and a user device 130 via a network 132. This communication enables the exchange of data, updates, and recommendations between the system 100 and the user. The computing device 102 and the user device 130 may include, but not limited to, a desktop computer, a smartphone, a laptop, a tablet, or any electronic device capable of executing the modules 108 of the system 100. The network 132 ensures real-time synchronization of data, which allows the users to receive timely updates, track their progress, and access their personalized oral care plans from any compatible device.

In one embodiment herein, the smart contract module 126 of the system 100 offers a compliance guarantee to the users. This guarantee applies exclusively to virgin teeth with no pre-existing oral diseases at the time of enrolment. The smart contract module 126 is configured to monitor the user's adherence to the personalized oral care plan and ensure that the user complies with brushing, plaque assessment, and microbiome testing protocols. In the event that a compliant user develops an oral disease despite following the prescribed oral care plan, the guarantee offers compensated treatment for the disease at no expense to the user. This embodiment underscores the system's commitment to preventive care and incentivizes compliance by providing an additional layer of trust and assurance to the users.

In another embodiment herein, the system 100 incorporates a cryptocurrency utility token to enhance user engagement and promote compliance with the personalized oral care plan. The utility token acts as an innovative incentive mechanism, which rewards the users for adherence to the personalized oral care plan and enables them to refer friends and family as potential subscribers. These referrals earn additional tokens, fostering a network effect for expanding a subscriber base. Furthermore, the system 100 allows third parties, such as sponsors or socially responsible organizations, to provide liquidity to the utility tokens in the form of fiat currency, discounts, or products. The third parties can gain publicity for their contributions to oral health while deriving social or commercial benefits. This embodiment combines financial incentives and community-building strategies to encourage user participation and promote the adoption of comprehensive oral care practices.

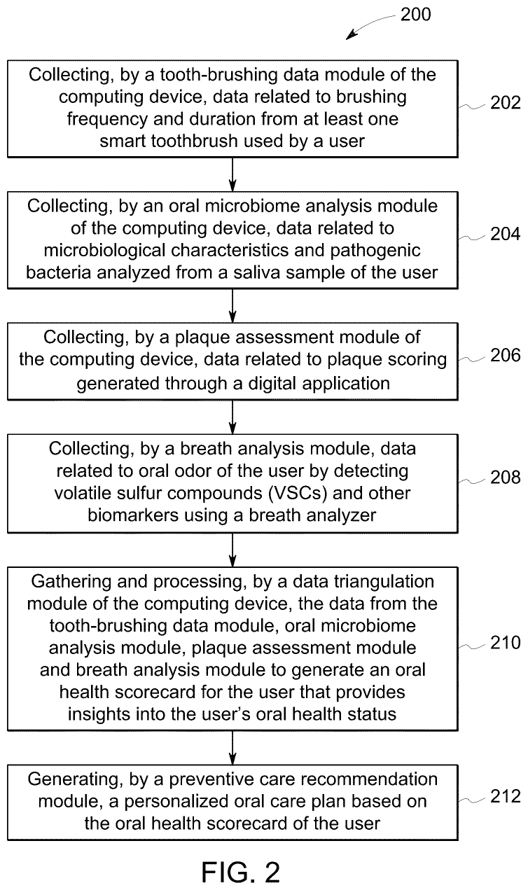

FIG. 2 refers to a flowchart 200 of a method for operating the system 100 for enhanced oral health and disease prevention. In one embodiment herein, the system 100 operates through a series of interconnected steps facilitated by the computing device 102. In the first step 202, the user initializes the system 100 by accessing the computing device 102. Once the system 100 is initialized, the user's tooth-brushing data is collected through the tooth-brushing data module 110. During this step, when the user brushes their teeth using the smart toothbrush 120, data such as brushing frequency and session duration is monitored and recorded. This recorded data is automatically transmitted to the brushing data module 110, which logs the user's brushing behavior.

At step 204, the user is instructed to collect a saliva sample using a saliva testing device 122. This saliva testing device 122 typically comprises a vial that is configured to collect the saliva sample in a sterile manner. The user collects the saliva sample as directed by the oral microbiome analysis module 112, thereby ensuring that the saliva sample is correctly stored for analysis. The saliva sample is then analyzed in a laboratory to identify microbiological characteristics and detect the presence of pathogenic bacteria, which provides valuable insights into the user's oral microbiome balance. This analyzed data related to the microbiological characteristics and the pathogenic bacteria can be uploaded directly to the oral microbiome analysis module 112.

Next, at step 206, the user is prompted to take images or videos of their teeth using the capturing unit. The plaque assessment module 114 guides the user on how to capture clear images or videos of their teeth from multiple angles, thereby ensuring that areas of the mouth typically missed during brushing are included. These images are uploaded to the digital application 124 installed in the user device 130, where advanced AI-based image processing models analyze the plaque levels on the user's teeth. Based on these images, the digital application 124 generates a plaque scoring that reflects the user's brushing effectiveness and identifies areas that may require improvement. This generated plaque scoring data is uploaded from the digital application 124 to the plaque assessment module 114.

Next, at step 208, the user is prompted to perform a breath analysis test using the breath analyzer 125 to assess their oral odor. The user exhales into the breath analyzer 125, which captures and analyzes the breath sample. The collected odor data is then transmitted to the breath analysis module 115, where AI-powered models process the results and classify the user's breath quality into predefined categories, such as normal, mild odor, moderate odor, or severe odor.

Once the data is collected by the tooth-brushing data module 110, the oral microbiome analysis module 112, the plaque assessment module 114 and the breath analysis module 115 is collected, the data triangulation module 116 processes and integrates all the collected data to generate an oral health scorecard for the user at step 210. The scorecard offers a comprehensive assessment of the user's oral health, which includes metrics such as brushing habits, microbial health, plaque build-up, and overall oral hygiene. The user can access their oral health scorecard via the user interface 134 of the computing device 102, thereby providing them with a visual summary of their oral health status and highlighting areas needing attention.

Based on the oral health scorecard, the preventive care recommendation module 118 generates a personalized oral care plan tailored to the user's specific needs at step 212. This personalized oral care plan may include recommendations for improved brushing techniques, dietary changes, or additional products (e.g., toothpaste or mouthwash). The personalized oral care plan also includes a suggested schedule for professional cleaning appointments with dental hygienists, which are customized to the user's oral health condition and needs. The user receives notifications or reminders via the computing device 102 and the user device 130 to ensure they follow the recommended schedule and appointments.

The smart contract module 126 of the system 100 ensures that the user adheres to their personalized oral care plan. The smart contract module 126 monitors the user's compliance with the oral care plan by automatically tracking their submission of data, such as toothbrush usage, saliva sample analysis, plaque assessment, and oral odor evaluation. The smart contract module 126 can also verify if the user has attended scheduled professional cleaning appointments and ensure that the user submits timely data. If the user meets the predefined conditions for compliance, the system 100 may offer incentives, such as discounts on the next subscription or rewards for consistent adherence. Conversely, if the user does not comply with the personalized oral care plan, the system 100 may apply penalties by increasing the cost of the personalized oral care plan or suggest further interventions to improve adherence.

Throughout this process, the computing device 102 communicates with the server 128 and the user device 130 via the network 132. This enables real-time synchronization of data across multiple devices, thereby allowing the user to access their health information, receive recommendations, and track their progress. The network 132 ensures that the modules 108 are updated automatically, and the system 100 continuously refines its recommendations based on the latest data collected. The user can access their personalized oral care plan and health scorecard using the computing device 102 and the user device 130, thereby enhancing the system's 100 usability and ensuring that the user remains engaged with their oral health goals.

In some embodiments, the system 100 may integrate three-dimensional (3D) scanning capabilities into the plaque assessment module 114 to provide a more comprehensive analysis of oral health. While traditional plaque detection focuses on surface-level build-up, the enhanced plaque assessment module 114 utilizes AI-powered 3D imaging to assess tooth wear, bite alignment, and early cavity formation. By capturing high-resolution scans of the user's teeth, the system 100 can detect subtle enamel erosion, improper occlusion patterns, and early-stage cavities that may not be visible through conventional methods. This advanced scanning functionality enhances preventive care by allowing the users and dental professionals to address emerging dental issues before they progress, thereby improving long-term oral health outcomes.

In some embodiments, the system 100 may implement blockchain-powered health records to securely store and manage oral microbiome data, plaque assessment results, brushing behavior patterns and oral odor scoring in a cloud-based data management platform. By leveraging decentralized ledger technology, the system 100 ensures tamper-proof, transparent, and secure storage of a user's oral health data, thereby preventing unauthorized modifications or breaches. This approach enables the users to maintain full control over their health records, thereby allowing selective and secure data sharing with dental professionals, insurance providers, or research institutions. Additionally, blockchain integration enhances compliance tracking by logging adherence to personalized oral care plans, thereby ensuring that the users follow recommended hygiene practices while providing verifiable proof of compliance for incentives or reduced treatment costs. The incorporation of the blockchain technology thus fosters data security, user privacy, and trust, transforming the way oral health information is managed and utilized.

In some embodiments, the smart toothbrush 120 may be equipped with real-time bacterial detection capabilities by utilizing integrated saliva sensors to analyze bacterial activity during brushing. The smart toothbrush 120 may continuously monitor salivary microbiome composition, thereby detecting the presence of harmful bacteria linked to oral diseases such as cavities, gingivitis, and periodontitis. This real-time data is instantly transmitted to the oral microbiome analysis module 112, where AI-driven models assess bacterial balance and provide instant feedback to the users via the user devices 130. By alerting the users to abnormal bacterial levels, the system 100 encourages timely intervention through optimized brushing techniques, targeted oral care recommendations, and, if necessary, professional dental consultations. This innovation enhances preventive oral care by offering proactive bacterial monitoring, ensuring that users maintain a healthier oral microbiome and reducing the risk of long-term dental issues. The system 100 may also include a QR code on the smart toothbrush 120 that links to a digital platform for further oral health analysis, scheduling reminders, and telemedicine consultations.

In another embodiment, the system 100 may incorporate a data-driven dental health curve model to further personalize treatment recommendations. By leveraging AI to analyze user dental data, such as brushing habits, microbiome composition, plaque levels, breath analysis, and imaging data, the system 100 can map the user's oral health status onto a predictive curve. This approach enables dynamic goal-setting, allowing the users to visualize their progress toward optimal oral health while receiving personalized recommendations designed to shift them to a healthier position on the curve. Additionally, AI-driven interpolation may be utilized to fill in missing data points, thereby ensuring more accurate assessments and tailored preventive strategies. This embodiment enhances patient motivation by providing a clear visual representation of potential improvements, further reinforcing compliance with the personalized oral care plan.

The system 100 enhances the oral health and prevents the oral diseases by integrating advanced technologies for monitoring, analysis, and personalized care planning. By leveraging data from the smart toothbrushes 120, the saliva testing devices 122, the digital applications 124 and the breath analyzer 125, the system 100 enables real-time tracking of oral hygiene practices, microbiological health, and plaque levels. This multi-faceted approach not only supports the users to gain actionable insights into their oral health but also encourages improved oral care habits through informed decision-making. Furthermore, the personalized oral care plans generated by the system 100, includes tailored schedules for professional cleanings, which offer a proactive strategy for maintaining optimal oral hygiene and preventing long-term dental issues.

Another significant advantage of the invention lies in its ability to promote user engagement and compliance through innovative features such as the smart contract module 126. This smart contract module 126 enforces adherence to personalized oral care plans by monitoring the submission of user data and user's attendance percentage at professional cleaning appointments while offering incentives or adjusting costs of the personalized oral care plans based on compliance. The system's 100 capability to analyze and process diverse data sources ensures accuracy and reliability in its recommendations, thereby fostering trust and usability. Additionally, the seamless communication between the computing device 102, the server 128, and the user device 130 via the network 132 ensures accessibility and convenience for the users, thereby making the system 100 a holistic and practical solution for improving oral health outcomes.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principles of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

It will readily be apparent that numerous modifications and alterations can be made to the processes described in the foregoing examples without departing from the principles underlying the invention, and all such modifications and alterations are intended to be embraced by this application.

The claimed invention is:

1. A system for enhanced oral health and disease prevention, comprising:
a computing device having a processor and a memory to store one or more instructions executable by said processor,
wherein said computing device is configured to execute a plurality of modules, wherein the plurality of modules includes:
a tooth-brushing data module configured to collect data related to brushing frequency and duration from at least one smart toothbrush used by a user;
an oral microbiome analysis module configured to collect data related to microbiological characteristics and pathogenic bacteria analyzed from a saliva sample of the user;
a plaque assessment module configured to collect data related to plaque scoring generated through a digital application;
a breath analysis module configured to collect data related to oral odor of the user by detecting volatile sulfur compounds (VSCs) and other biomarkers using a breath analyzer;
a data triangulation module configured to gather and process the data from said tooth-brushing data module, oral microbiome analysis module, plaque assessment module, and breath analysis module to generate an oral health scorecard for the user that provides insights into the user's oral health status; and
a preventive care recommendation module configured to generate a personalized oral care plan based on the oral health scorecard of the user, wherein the personalized oral care plan includes a tailored schedule of professional cleaning appointments with dental hygienists, thereby enhancing oral health and disease prevention.

2. The system for enhanced oral health and disease prevention of claim 1, wherein said at least one smart toothbrush is a manually operated toothbrush equipped with a clip-on attachment that monitors and records the brushing frequency and duration.

3. The system for enhanced oral health and disease prevention of claim 1, wherein the saliva sample is collected by the user in a vial provided by a saliva testing device and subsequently analyzed in a laboratory to identify microbiological characteristics and detect the presence of pathogenic bacteria.

4. The system for enhanced oral health and disease prevention of claim 1, wherein said digital application uses one or more advanced artificial intelligence (AI)-based image processing models that are configured to analyze one or more images or videos of the user's teeth captured by at least one capturing unit to generate the plaque scoring, which evaluates effectiveness of the user's tooth-brushing technique.

5. The system for enhanced oral health and disease prevention of claim 4, wherein said at least one capturing unit includes a camera, a smartphone, a webcam, an intraoral camera, an intraoral scanner, a handheld imaging device and thereof.

6. The system for enhanced oral health and disease prevention of claim 1, wherein said plurality of modules further comprises a smart contract module that is configured to enforce user's compliance with the personalized oral care plan by automatically monitoring and verifying the user's submission of data from said at least one smart toothbrush, the at least one saliva testing device, the digital application, the breath analyzer and user's attendance percentage at scheduled professional cleaning appointments.

7. The system for enhanced oral health and disease prevention of claim 6, wherein said smart contract module is further configured to adjust cost of the personalized oral care plan based on the user's compliance.

8. The system for enhanced oral health and disease prevention of claim 1, wherein said computing device is configured to communicate with a server and a user device via a network, wherein said computing device and said user device include at least one of a computer, a smartphone, a laptop, a tablet, and thereof.

9. A method of operating a system for enhanced oral health and disease prevention using a computing device, comprising:
collecting, by a tooth-brushing data module of the computing device, data related to brushing frequency and duration from at least one smart toothbrush used by a user;
collecting, by an oral microbiome analysis module of the computing device, data related to microbiological characteristics and pathogenic bacteria analyzed from a saliva sample of the user;
collecting, by a plaque assessment module of the computing device, data related to plaque scoring generated through a digital application;
collecting, by a breath analysis module, data related to oral odor of the user by detecting volatile sulfur compounds (VSCs) and other biomarkers using a breath analyzer;
gathering and processing, by a data triangulation module of the computing device, the data from said tooth-brushing data module, oral microbiome analysis module, plaque assessment module and breath analysis module to generate an oral health scorecard for the user that provides insights into the user's oral health status; and
generating, by a preventive care recommendation module, a personalized oral care plan based on the oral health scorecard of the user.

10. The method of claim 9, wherein the personalized oral care plan includes a tailored schedule of professional cleaning appointments with dental hygienists for enhanced oral health and disease prevention.

11. The method of claim 9, wherein said at least one smart toothbrush is a manually operated toothbrush equipped with a clip-on attachment that monitors and records the brushing frequency and duration.

12. The method of claim 9, wherein said saliva sample is collected by the user in a vial provided by a saliva testing device and subsequently analyzed in a laboratory to identify microbiological characteristics and detect the presence of pathogenic bacteria.

13. The method of claim 9, wherein said digital application uses one or more advanced artificial intelligence (AI)-based image processing models that are configured to analyze one or more images or videos of the user's teeth captured by at least one capturing unit to generate the plaque scoring, which evaluates effectiveness of the user's tooth-brushing technique.

14. The method of claim 13, wherein said at least one capturing unit includes a camera, a smartphone, a webcam, an intraoral camera, an intraoral scanner, a handheld imaging device and thereof.

15. The method of claim 9, wherein said plurality of modules further comprises a smart contract module that is configured to enforce user's compliance with the personalized oral care plan by automatically monitoring and verifying the user's submission of data from said at least one smart toothbrush, the at least one saliva testing device, the digital application, the breath analyzer and user's attendance percentage at scheduled professional cleaning appointments.

16. The method of claim 15, wherein said smart contract module is further configured to adjust cost of the personalized oral care plan based on the user's compliance.

17. The method of claim 9, wherein said computing device is configured to communicate with a server and a user device via a network, wherein said computing device and said user device include at least one of a computer, a smartphone, a laptop, a tablet, and thereof.
